(19) United States
(12) Patent Application Publication
(10) Pub. No.: US 2014/0245814 A1
(43) Pub. Date: Sep. 4, 2014

(54) DEVICE FOR MEASURING THE CONCENTRATION OF A GAS DISSOLVED IN AN ELECTRICAL INSULATION OIL

(71) Applicant: TECHIMP TECHNOLOGIES S.R.L., BOLOGNA (IT)

(72) Inventors: Gian Carlo Montanari, Casalecchio Di Reno (BO) (IT); Stefano Serra, San Vittore Olona (MI) (IT); Paolo Francia, Zola Predosa (BO) (IT)

(73) Assignee: Techimp Technologies S.R.L., Bologna (IT)

(21) Appl. No.: 14/353,175

(22) PCT Filed: Nov. 13, 2012
(86) PCT No.: PCT/IB2012/056388

§ 371 Date: Apr. 21, 2014

(30) Foreign Application Priority Data
Nov. 23, 2011 (IT) BO2011A000670

(51) Int. Cl. G01N 33/28

(52) U.S. Cl. CPC G01N 33/28
USPC 73/19.1; 29/890.09

(57) ABSTRACT

A device for measuring the concentration of a gas dissolved in an electrical insulation oil comprises a measuring member (2), internally defining a measuring chamber (3) and a conduit (4) to access the measuring chamber (3), a membrane (6) permeable to gases, inserted inside the conduit (4) for insulating the measuring chamber (3) against the oil and allowing gas to pass from the oil to the measuring chamber (3), a sensor (20) located in the measuring chamber (3) for measuring the concentration of gas in the measuring chamber (3), and a reinforcing grid (8) positioned in the conduit (4) for preventing deformation of the membrane (6), where the grid (8) is located at a position adjacent to the membrane (6) on the side opposite to the measuring chamber (3), and has at least one through section (9, 10) for allowing the oil to exert pressure directly on the membrane (6).

### TECHNICAL FIELD

The object of this invention is a device for measuring the concentration of a gas dissolved in an electrical insulation oil.

More generally, this invention relates to a diagnostic device for assessing the insulation condition of an electrical insulation oil of electrical equipment.

The invention thus addresses the field of diagnostic assessment of oil-insulated electrical equipment such as transformers or cables.

### BACKGROUND ART

Partial discharge is a well-known phenomenon in oil-insulated electrical equipment subjected to medium or high voltages.

A partial discharge is an electric discharge limited to a portion of the insulation of an electrical system and does not therefore cause immediate failure of the system but, more generally, causes its gradual degradation.

By their very nature, therefore, partial discharges are substantially limited to a defect in the insulating system.

In light of this, the use of a liquid insulator such as oil has the advantage of allowing convective movements within the oil and thanks to certain chemical processes, this type of insulation is at least partly self-restorative, that is to say, it is capable of at least partly compensating the degradation it undergoes during the operation of the transformer.

It is known that partial discharges that take place in the oil cause gases to be formed.

A further gas generating factor is that the oil reaches particularly high temperatures.

For this reason, diagnostic systems for assessing the insulation condition of oil-insulated transformers have been in use for some time. These systems are based on the assessment of the concentration of gases in the oil and on the analysis of said gases.

More advanced solutions in this field involve the use of a membrane impermeable to oil but permeable to gas, which is interposed between an oil container (connected to the insulating oil by means of a specific offtake) and a measuring chamber containing only gas; an example of said technical solution is described in US2006/032742. The measuring chamber receives through the membrane a part of the gas present in the oil.

That way, by separating the measuring chamber from the oil it is possible to place a sensor in the measuring chamber to measure the value of gas concentration in the measuring chamber. The sensor is particularly reliable because it is never in contact with the oil and has the advantage of allowing constant monitoring of the composition and concentration of the gases in the oil.

Furthermore, it has been observed that the membrane is very sensitive to pressure jumps in the oil circuit due, for example, to failures, breakdowns, thermal shocks, maintenance operations.

In light of this, it should be noted that the pressure jumps may be positive (overpressure of the oil) or negative (underpressure of the oil) as compared to a reference pressure value of the oil, according to which the device is set up and the membrane is calibrated.

To counter the positive pressure jumps, that is, the overpressures in the oil, it is possible to place a disc made of porous material (e.g. sintered bronze) in the gas chamber, in contact with the membrane, to achieve a uniform supporting surface for the membrane, which the latter may lie on when pushed by an overpressure of the oil.

Such a solution is disclosed, e.g. in patent document GB2053482.

This porous disc, however, is not capable of exerting any supporting action for the membrane when the latter is sucked in on the oil side when an underpressure is generated in the oil.

Moreover, negative pressure jumps, that is, underpressure in the oil, constitute a particularly dangerous circumstance for the device.

Indeed, the suction action on the membrane may cause it to break or cause it to withdraw from its proper housing, so that oil may leak into the measuring chamber, and damage may also be caused to the electrical part thereof.

To overcome these problems, the use is known of radically different diagnostic systems, which do not involve the use of the membrane and instead involve collecting oil samples to cause the dissolved gases to evaporate and hence to apply chemical-physical (chromatographic) measuring techniques.

These latter diagnostic systems, however, are inevitably quite complex and costly and furthermore, may not be applied on site to monitor the electrical equipment during the operation thereof because they must be applied in a laboratory. Thus, these systems introduce further drawbacks. DE19503802 discloses a protective shield which completely surrounds the membrane, on both sides of the membrane.

However, said solution affects the performance of the membrane.

### DISCLOSURE OF THE INVENTION

The aim of this invention is to provide a device for measuring the concentration of a gas dissolved in an electrical insulation oil which overcomes the above-mentioned drawbacks of the known art.

In particular, this invention has for an aim to provide a device for measuring the concentration of a gas dissolved in an electrical insulation oil of electrical equipment, which is highly reliable in particular reference to resistance to the negative pressure jumps which occur in the oil.

Another aim of the invention is to provide a device for measuring the concentration of a gas dissolved in an electrical insulation oil (and a method for obtaining it) which is particularly robust and accurate.

These aims are fully achieved by the device and method according to the invention as characterized in the appended claims.

It should be noted that the device according to the invention is, in particular, a device for measuring the concentration of a gas dissolved in an electrical insulation oil of a transformer (insulated with paper and oil).

The device might, however, also be applied to other equipment, for example cables insulated with impregnated paper and tap changer devices.

In particular, the device comprises:
a measuring member, with a measuring chamber and a conduit communicating with said measuring chamber, said conduit defining a termination which is connectable to a container for the electrical insulation oil of said equipment;
a membrane permeable to gases, inserted inside the conduit, for separating the measuring chamber from the oil in said container and for allowing gas to pass from the oil towards the measuring chamber;
one or more sensors located in the measuring chamber for measuring concentration values of gas in the measuring chamber.

According to the invention, the device (and in particular said measuring membrane) comprises a reinforcing element located in the conduit at a position adjacent to a surface of the membrane facing the side opposite to the measuring chamber (and hence operatively immersed in the oil when the device is coupled with the electrical equipment), and having a plurality of resistant portions defining through apertures; said through apertures form a section through which the oil passes and comes operatively in contact with said surface of the membrane.

Preferably, the reinforcing element is located only at the side opposite to the measuring chamber; hence, preferably, no reinforcing element faces the membrane at the side of the measuring chamber. This improves the performance of the device.

Said reinforcing element is preferably in direct contact with the membrane.

Said reinforcing element is preferably rigid.

Thus, the reinforcing element constitutes an obstacle (by means of a mechanical constraint) to deformation of the membrane in a direction opposite to the measuring chamber (also called gas chamber) when the membrane is subjected to a suction action by the oil (when there is a negative pressure jump in the oil circuit of the electrical equipment).

On the other hand, the conformation of the reinforcing element, which defines a predetermined through section, ensures the device is efficient and sensitive because the oil is in contact with a surface of the membrane which is large enough to allow the gas dissolved in the oil to pass through the membrane and reach the measuring chamber.

The reinforcing element is preferably grid shaped and extends about an axis, said through apertures being angularly spaced (preferably uniformly spaced) about said axis.

In light of this, the resistant portions constitute arms forming the grid.

In light of this, it should be noted that the reinforcing element comprises a plurality of intersecting zones of the resistant portions (that is, zones in which the resistant portions meet and are joined).

The reinforcing element preferably has a ring shaped resistant portion (preferably, circular, alternatively elliptical shaped or in any event defined by a closed curved line) which is positioned centrally (that is, adjacent to a middle portion of the membrane).

Therefore, said resistant middle position defines a middle aperture of the reinforcing element.

Said constructive features of the reinforcing element allow the surface occupied by the reinforcing element to be minimized (in the plane defined by said side of the membrane) while also allowing the reinforcing element to exert an effective mechanical constraining action thus minimizing the deformation of the membrane when there is a negative pressure in the oil. The resistant portions (that is, the arms) of the reinforcing element preferably have a minimum dimension (measured in any direction along the plane the membrane lies in) of at least 2 mm.

Furthermore, said resistant portions preferably define rounded or radiused side edges in a zone of the reinforcing element adjacent to said surface of the membrane.

Also, preferably, the reinforcing element is shaped so that the edges defined by the intersections of the resistant portions (at said intersection zones) are radiused, that is, they are not sharp edges.

Said features (minimum thickness of the grid arms greater than a reference value and no sharp edges in the grid portion operatively in contact with the membrane) make the device particularly robust and reliable, while preventing the membrane from being damaged due to a pressure thereof against the reinforcing element (when there are negative pressure jumps in the oil).

Furthermore, the reinforcing element is preferably made in a single piece with said measuring member.

This makes the device particularly safe with respect to the risk of any oil infiltrations because it avoids the need for sealing gaskets for the reinforcing element.

The invention also provides a method for making a device for measuring the concentration of a gas dissolved in an electrical insulation oil of electrical equipment (in particular a transformer).

This method comprises a step of preparing a measuring member, with a measuring chamber and a conduit communicating with said measuring chamber, said conduit defining a termination which is connectable to a container for the electrical insulation oil of said equipment; a membrane permeable to gases, inserted inside the conduit, for separating the measuring chamber from the oil in said container and for allowing gas to pass from the oil towards the measuring chamber; a sensor located in the measuring chamber for measuring the concentration values of gas in the measuring chamber.

According to the invention, the method comprises a step of positioning a reinforcing element in the conduit at a position adjacent to a surface of the membrane facing the side opposite to the measuring chamber, said reinforcing element having a plurality of resistant portions defining between them through apertures which form a section through which the oil passes and comes operatively in contact with said surface of the membrane.

The reinforcing element is preferably made in a single piece with at least one portion of the measuring member.

In light of this, the method preferably comprises the following steps:
preparing said at least one portion of the measuring member so that it at least partly defines said conduit and having, inside said conduit, a wall at least partly obstructing the conduit made in a single piece with said portion of the measuring member;
performing, on said wall, mechanical drilling to obtain said resistant portions and said through apertures.

The method also preferably comprises a successive step of mechanically rounding said resistant portions (that is, the arms of the grid) to remove sharp edges from portions of the reinforcing element facing the membrane and operatively in contact therewith when the membrane is subjected to a suction action by the oil.

### BRIEF DESCRIPTION OF DRAWINGS

This and other features of the invention will become more apparent from the following description of a preferred, non-limiting example embodiment of it, with reference to the accompanying drawings, in which:

FIG. 1 depicts a sectional view of a device according to this invention;

FIG. 2 depicts a sectional view of a part of the device of FIG. 1;

FIG. 3 shows a component of the device of FIG. 1;

FIG. 4 shows a front view of a detail of the component of FIG. 3;

FIG. 5A is a sectional view of the component of FIG. 3 according to the directrix V-V of FIG. 4;

FIG. 5B shows an enlarged detail of the view of FIG. 5A;

### DETAILED DESCRIPTION OF PREFERRED EMBODIMENTS OF THE INVENTION

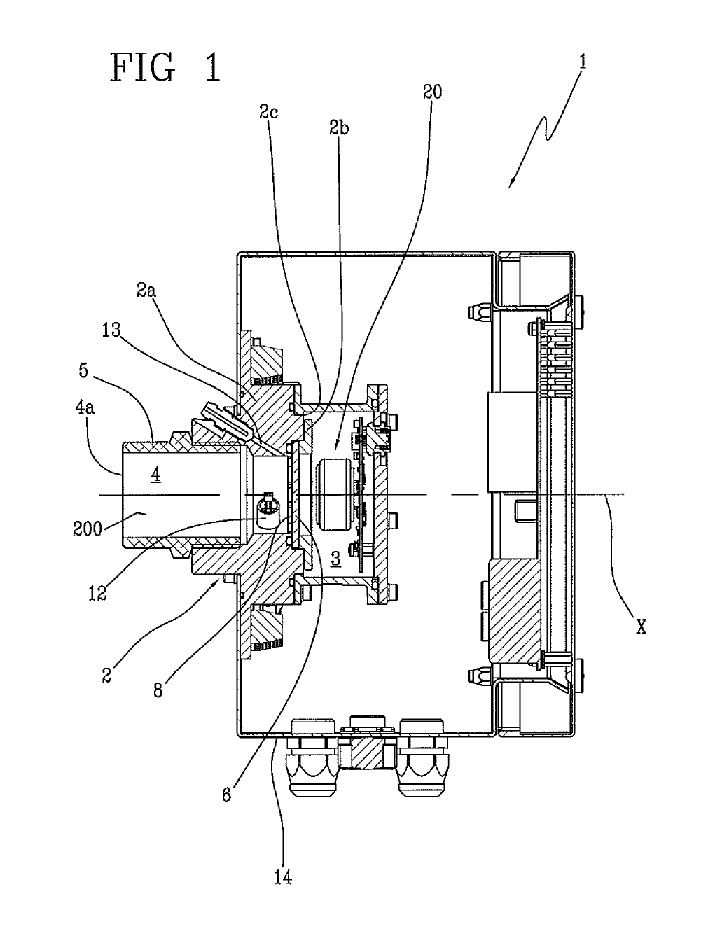

The numeral 1 in FIG. 1 denotes a device according to this invention.

The device 1 is a device for measuring the concentration of a gas dissolved in an electrical insulation oil of electrical equipment, for example a transformer for medium or high voltages.

More specifically, the device 1 is a device for deriving the concentration of a gas dissolved in the insulation oil 200 of electrical equipment 3 (typically a transformer but possibly also a cable or other oil-insulated electrical equipment).

Derivation of this kind is preliminary to analysis of the gases dissolved in the oil 200, called DGA (dissolved gas analysis).

As is known, this type of analysis (DGA) is used for diagnostic purposes for deriving information about the condition of the insulation of the electrical equipment 3.

Indeed, gases dissolved in the oil 200 are generated by partial discharges occurring in the oil (or in parts of the electrical equipment 3 subjected to the electric field and to contact with the oil 200) or by overheating of the oil 200; both said circumstances constitute possible causes of breakdown or in any event are a sign of risk for the operativeness of the electrical equipment.

The electrical equipment (which is not shown in the drawings because it is not part of the invention) comprises a generic container for the electrical insulating oil, typically an oil circulating circuit, equipped with an offtake through which a portion of the oil may be collected and analysis performed to measure the concentration of the gases dissolved therein.

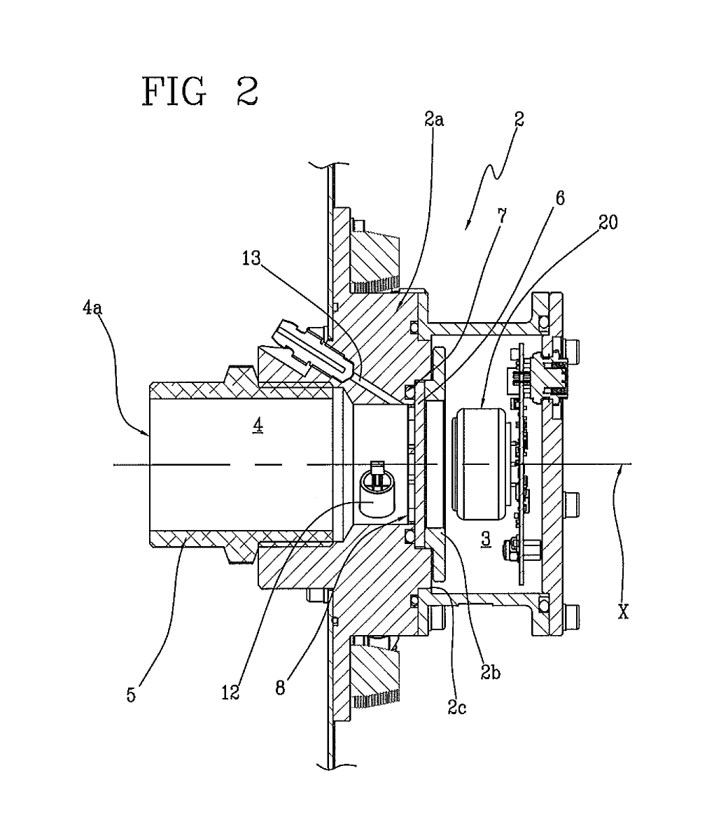

As shown in FIG. 1 and in greater detail in FIG. 2, the device 1 comprises a measuring member 2, which internally defines a measuring chamber 3 and a conduit 4 for accessing the measuring chamber 3.

In particular, the conduit 4 has a termination 4a which is connectable to a container of the electrical insulating oil (typically an offtake section of an oil circuit) and leads into the measuring chamber 3 on the side opposite to the termination.

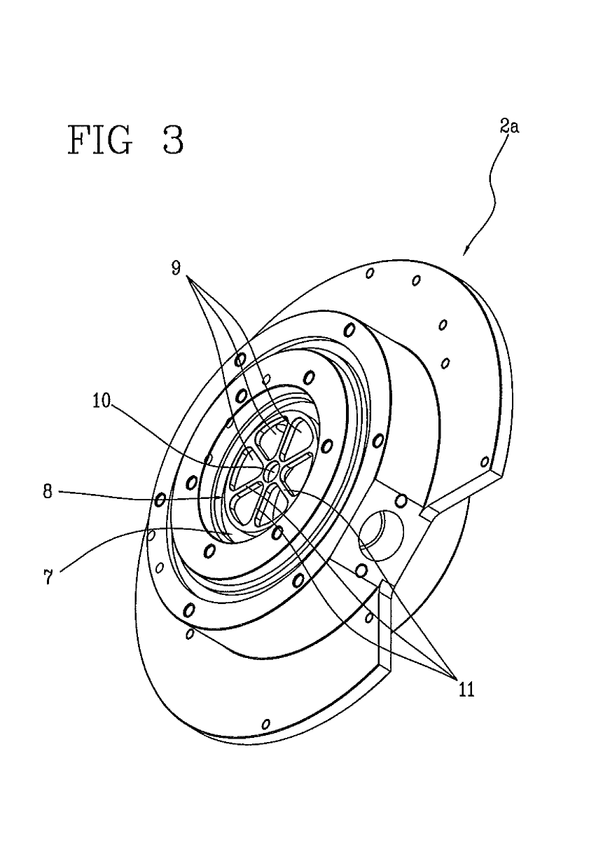

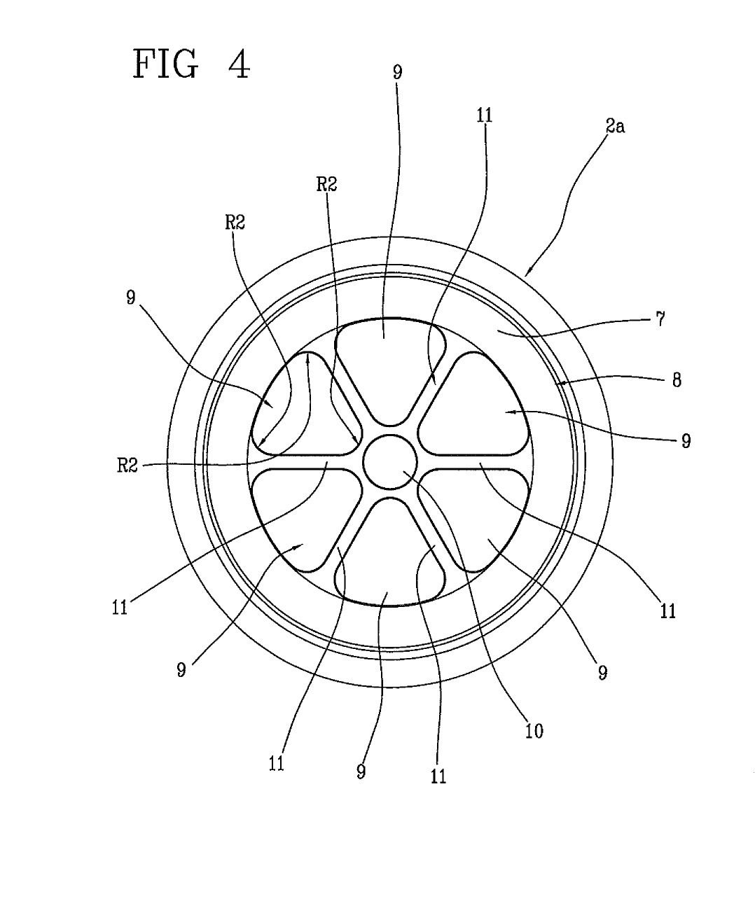

The conduit is preferably at least in part defined by a first part 2a of the measuring member, which in the embodiment shown in the accompanying drawings and clearly visible in FIG. 3, is ring- or disc-shaped and extends about an axis “X” defining the axis of the conduit 4.

The first part 2a of the measuring member 2 on the side opposite to the measuring chamber 3 is preferably associated with a connecting terminal 5 which is coaxial to the axis “X” and defines an extension of the conduit 4 up to the termination 4a. The connecting terminal 5 is stably connected to the first part 2a of the measuring member 2, for example through threading.

Inside the conduit 4 there is a membrane 6 permeable to gases, whose function is to insulate the measuring chamber 3 against the oil in the oil circuit and at the same time, allow the gas to pass from the oil to the measuring chamber 3.

Thanks to the use of the membrane 6, the measuring chamber is insulated against the oil and is only intended to receive the gas which originates from partial discharges or overheating in the oil and which passes through the membrane 6.

Thus, a sensor 20 for measuring the concentration of one or more gases in the measuring chamber 3 may be located inside the measuring chamber.

The sensor 6 can measure the concentration of one or more predetermined types of gas, preferably, but not limited to, the following:
carbon monoxide, denoted CO;
hydrogen, denoted H2;
carbon dioxide, denoted CO2;
acetylene, denoted C2H2;
ethylene, denoted C2H4;
methane, denoted CH4.

Alternatively, instead of a single sensor, the device 1 might comprise a plurality of sensors, each designed to measure the concentration of a predetermined type of gas.

The device 1 also comprises a control unit (not illustrated) or a processor or any other processing means, electrically connected to the sensor 20 to receive from the latter a signal corresponding to the value/values of concentration of the predetermined type/types of gas measured in the measuring chamber 3 and to process that signal according to known types of algorithms.

The membrane 6, which is elastically deformable and preferably made of Teflon and/or copolymers deriving therefrom, lies in a plane perpendicular to the axis “X”.

The membrane 6 is preferably located in the conduit 4 at a position which is offset towards the measuring chamber 3, and in particular, at an annular groove 7 made on a front surface 2c of the first part 2a of the measuring member 2 facing towards the measuring chamber 3 (FIG. 3).

The measuring member 2 also comprises a second part 2b, which can be stably abutted (for example, by means of connection with threaded members) to the above-mentioned front surface 2c. Hence, said second part 2b of the measuring member 2 defines a housing for the membrane 6 in cooperation with the first part 2a.

Furthermore, the second part 2b of the measuring member 2 preferably comprises a full disc at least partly made of porous material, preferably sintered bronze, which is permeable to at least one type of gas dissolved in the oil and whose function is to prevent deformation of the membrane towards the measuring chamber 3.

In other words, the disc of porous material makes a continuous constraining surface for the membrane on which the latter may lie when pressed on from the opposite side by the pressure of the oil coming from the oil circulating circuit.

Advantageously, the measuring member 2 also comprises a reinforcing element 8 positioned in the conduit 4 also to prevent a deformation of the membrane 6 in the direction opposite to the measuring chamber 3, that is, a deformation induced by a negative pressure transmitted by the oil.

The reinforcing element 8 is located at a position adjacent to a surface of the membrane facing the “oil side”, that is, facing the side opposite to the measuring chamber 3.

Preferably, the reinforcing element 8 lies mainly in a plane which is perpendicular to the axis “X” and is hence parallel to the plane in which the membrane 6 lies.

Advantageously, the reinforcing element 8 also has at least one section for allowing the oil to pass to exert pressure directly on the above-mentioned surface of the membrane 6.

In a preferred embodiment and as shown in the accompanying drawings, the reinforcing element 8 is obtained by means of a grid and the section for the oil to pass is defined by a plurality of through apertures 9 for the oil, which are angularly spaced (preferably equally spaced) about the axis “X”. There are preferably six through apertures 9 arranged about the axis “X” and they substantially take on the shape of an equilateral triangle.

Surprisingly, the above-mentioned grid structure of the reinforcing element 8 is particularly effective in constraining the membrane 6, that is, in preventing deformations thereof under the suction action by the oil, and at the same time allows the membrane 6 to make contact with the oil on a sufficiently large surface so as to adequately allow gas to pass from the oil to the measuring chamber 3, thus ensuring increased precision and measuring sensitivity.

The reinforcing element 8 also preferably has a middle aperture 10 centred on the axis “X”.

Said aperture serves the function of further improving the effectiveness of the gas passing through the membrane 6 by acting in such a way as to allow an action by the oil on the membrane 6 at a portion of the latter which is more effective for the passage of gases.

Furthermore, said middle aperture 10 serves the function of increasing the resistance to negative pressures by constraining the membrane at the point of maximum mechanical stress.

Between them, the through apertures 9 identify respective resistant portions of the reinforcing element 8 which serve the function of constraining against the deformation of membrane 6. In the embodiment described, said resistant portions are defined by a plurality of radial arms 11 which define the above-mentioned through apertures for the oil.

The reinforcing element 8 (grid) is made from rigid material, preferably metal (for example, anodized aluminium).

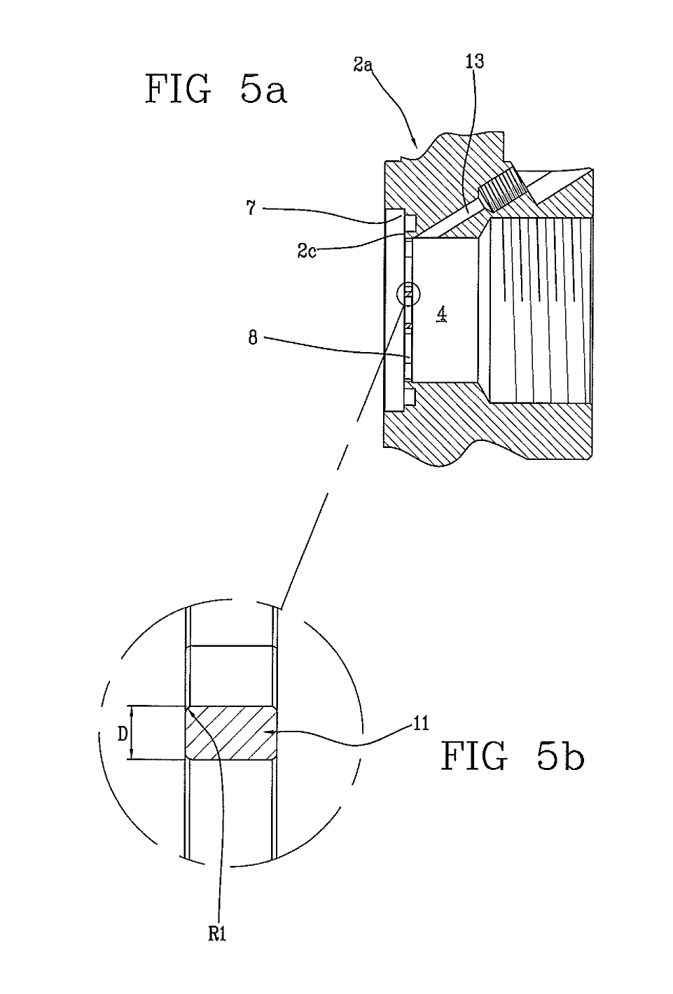

In order to allow an excellent mechanical interaction between the membrane 6 and the radial arms 11 of the reinforcing element 8, the radial arms 11 preferably have side edges which are rounded or radiused according to a plane perpendicular to the direction of radial extension of the arms 11, as shown in detail in FIG. 5B. This prevents the side edges of the radial arms 11 from cutting into or damaging the membrane 6 when it is pressed against the reinforcing element 8.

The radius “R1” of said edges is preferably within the range from 0.5 mm to 2 mm and, more preferably, it is approximately 1 mm.

Further, for the same reason, the reinforcing element 8 has, at least in one portion thereof adapted to receive the membrane 6, radiused edges on the above-mentioned plane of the reinforcing element 8, preferably with minimum upper radius “R2” within the range from 2 mm to 15 mm; the minimum radius “R2” is more particularly approximately 5 mm.

Again for the same reason, it is just as preferable that the minimum dimension “D” (which is measured along a plane parallel to the plane in which the membrane 6 lies) of the above-mentioned resistant portions (regardless of whether or not they are defined by the mentioned radial arms 11 or by other embodiments) be equal to or greater than 2 mm.

In a preferred embodiment, and as shown in the accompanying drawings, the reinforcing element 8 is made in a single piece with the first part 2a of the measuring member 2.

In this embodiment, the reinforcing element 8 may be obtained by means of the following technological process:
preparing the above-mentioned first portion 2a of the measuring member 2, which already has inside it (hence inside the conduit 4) a wall which at least partly obstructs the conduit 4, made in a single piece with the first portion 2a of the measuring member 2;
performing, on said wall, mechanical drilling and/or radiusing to obtain at least one through section in the wall to make the reinforcing element 8.

As shown in FIGS. 1 and 2, the measuring member 2 also has an oil temperature and moisture sensor 12, which extends inside the conduit 4 and is positioned near the membrane 6.

The measuring member 2 may also have a bleed channel 13 for the air, made on the first part 2a and to be used to bleed the air from the oil circuit during installation of the device.

Moreover as shown in FIG. 1, the device 1 comprises a protective cover 14, which is connected to the measuring member 2 (and in particular to the first part 2a thereof) and in addition to the various terminals and external contacts, contains the electronic components needed for the functions of storing data, communication, generating alarms and processing for diagnostic purposes the signal generated by the sensor 20.

It will be understood that the invention described may be useful in many industrial applications and may be modified and adapted in several ways without thereby departing from the scope of the inventive concept. Moreover, all the details of the invention may be substituted by technically equivalent elements.

1. A device for measuring the concentration of a gas dissolved in an electrical insulation oil, comprising:
a measuring member (2), with a measuring chamber (3) and a conduit (4) communicating with said measuring chamber (3), said conduit (4) defining a termination (4a) which is connectable to a container for the electrical insulation oil of said equipment;
a membrane (6) permeable to gases, inserted inside the conduit (4), for separating the measuring chamber (3) from the oil in said container and for allowing gas to pass from the oil towards the measuring chamber (3);
a sensor (20) located in the measuring chamber (3) to measure a value of the gas concentration in the measuring chamber (3),
wherein said measuring member (2) comprises a reinforcing element (8), located in the conduit (4) at a position adjacent to a surface of the membrane (6) facing the side opposite to the measuring chamber (3), and having a plurality of resistant portions (11) defining between them through apertures (9), which form a section (9, 10) through which the oil passes and comes operatively in contact with said surface of the membrane (6).

2. A device according to claim 1, wherein said reinforcing element (8) is grid shaped and extends about an axis (X), said through apertures (9) being angularly spaced on said grid (8) about said axis (X).

3. A device according to claim 2, comprising a plurality of through apertures (9) which are uniformly spaced and separated by corresponding resistant portions (11) which are radially arranged.

4. A device according to claim 1, wherein said reinforcing element (8) has a ring shaped resistant portion which is positioned centrally, and defining a middle aperture (10) of the reinforcing element (8).

5. A device according to claim 1, wherein said reinforcing element (8) is rigid.

6. A device according to claim 1, wherein said resistant portions (11) have a minimum dimension (D) of at least 2 mm, which is measured in any one direction along a plane in which the membrane (6) lies.

7. A device according to claim 1, wherein said resistant portions (11) define rounded or radiused side edges in a zone of the reinforcing element (8) adjacent to said surface of the membrane (6).

8. A device according to claim 1, wherein the reinforcing element (8) has a plurality of intersecting zones of the resistant portions (11) and is shaped so that the edges defined by said intersections are radiused, without corners.

9. A device according to claim 1, wherein said measuring member (2) comprises at least a first part (2a) defining at least one section of said conduit (4) and wherein the reinforcing element (8) is made in a single piece with said first part (2a).

10. A device according to claim 9, wherein said measuring member (2) comprises a second part (2b) able to be abutted to said first part (2a) and defining a seat (7) for housing the membrane (6) in cooperation with the first part (2a), said second part (2b) comprising a full disc, at least partly made of porous material, which is permeable to gases and which is located at a position adjacent to a surface of the membrane (6) facing towards the measuring chamber (3), for preventing deformation of the membrane (6) towards the measuring chamber (3).

11. A method for making a device for measuring the concentration of a gas dissolved in an electrical insulation oil of electrical equipment, comprising the following steps:
preparing a measuring member (2), with a measuring chamber (3) and a conduit (4) communicating with said measuring chamber (3), said conduit (4) defining a termination (4a) which is connectable to a container for the electrical insulation oil of said equipment; a membrane (6) permeable to gases, inserted inside the conduit (4), for separating the measuring chamber (3) from the oil in said container and for allowing gas to pass from the oil towards the measuring chamber (3); a sensor (20) located in the measuring chamber (3) for measuring the concentration values of gas in the measuring chamber (3);
positioning a reinforcing element (8) in the conduit (4) at a position adjacent to a surface of the membrane (6) facing the side opposite to the measuring chamber (3), said reinforcing element (8) having a plurality of resistant portions (11) defining between them through apertures (9) which form a section (9, 10) through which the oil passes and comes operatively in contact with said surface of the membrane (6).

12. A method according to claim 11, wherein said reinforcing element (8) is made in a single piece with at least one portion (2a) of the measuring member (2).

13. A method according to claim 12, comprising the following steps:
preparing said at least one portion (2a) of the measuring member (2) so that it at least partly defines said conduit (4) and having, inside said conduit (4), a wall which at least partly obstructs the conduit (4) made in a single piece with said portion (2a) of the measuring member (2);
performing, on said wall, mechanical drilling to obtain said resistant portions (11) and said through apertures (9).

14. A method according to claim 13, comprising mechanical radiusing of said resistant portions (11), to remove sharp edges from portions of the reinforcing element (8) facing the membrane and operatively in contact therewith when the membrane is subjected to a suction action by the oil.
